(19) United States
(12) Patent Application Publication
(10) Pub. No.: US 2017/0231547 A1
(43) Pub. Date: Aug. 17, 2017

(54) APPARATUS AND METHODS FOR SCREENING PATIENTS FOR BLADDER CONTROL VIA PUDENDAL NERVE STIMULATION

(71) Applicant: Incube Labs, LLC, San Jose, CA (US)

(72) Inventor: Mir Imran, Los Altos Hills, CA (US)

(73) Assignee: Incube Labs, LLC, San Jose, CA (US)

(21) Appl. No.: 15/431,621

(22) Filed: Feb. 13, 2017

Related U.S. Application Data
(63) Continuation-in-part of application No. 15/153,314, filed on May 12, 2016.
(60) Provisional application No. 62/294,613, filed on Feb. 12, 2016.

(51) Int. Cl. A61B 5/20; A61N 1/05; A61N 1/36; A61B 5/00

(52) U.S. Cl. CPC A61B 5/205; A61B 5/6853; A61N 1/0514; A61N 1/0551; A61N 1/36007

(57) ABSTRACT

Embodiments of the invention provide apparatus and methods for testing the efficacy of nerve stimulation therapy to treat patients who have urinary dysfunction prior to the implantation of an apparatus to treat the dysfunction. The apparatus and methods provide means to selectively stimulate the pudendal nerve with high and low frequency current to produce a physiologic response involved in the urination process (e.g., relaxation of the urinary sphincter and contraction of the bladder) and then measure information relating to the response. Particular embodiments involve the introduction of a urethral catheter configured to both fill the bladder and test the ability to control bladder voiding by applying stimulation current to the pudendal nerve and then measure the response information such as bladder pressure, urinary sphincter pressure and urinary flow rate. The catheter can include at least two electrodes and separate pressure sensors positioned for measuring the urinary sphincter and bladder pressure.

### CROSS-REFERENCE TO RELATED APPLICATIONS

This application claims the benefit of priority to U.S. Provisional Application No. 62/294,613, (Attorney Docket No. 42197-739.101), filed Feb. 12, 2016, the entire content of which is fully incorporated herein by reference.

This application also claims the benefit of priority to U.S. patent application Ser. No. 15/153,314, filed May 12, 2016, which is incorporated by reference herein for all purposes. This application is also related to U.S. patent application Ser. No. 15/410,692, filed Jan. 19, 2017 which is incorporated by reference herein for all purposes.

### BACKGROUND OF THE INVENTION

1. Field of the Invention

The present invention relates to medical apparatus and methods. More particularly, the present invent relates to systems and methods for screening paralyzed and incontinent patients to determine if they can benefit from pudendal nerve stimulation to control urinary function.

Many disorders can result in loss of a patient's ability to voluntarily control bladder function. Most commonly, patients suffering from spinal cord injuries can lose not only the ability to voluntarily control urination, but also the ability to sense when the bladder is full. Such patients have usually had to rely on the chronic use of a urinary drainage (Foley) catheter which is placed through the urethra and has a distal tip residing in the bladder. Such Foley catheters present a constant risk of infection which is exacerbated by the frequent need to exchange a clogged catheter for a new catheter. Moreover, Foley catheters usually drain into a bag which the patient must carry when away from home or a treatment facility. The need to carry the drain bag is a significant burden to many patients.

To at least partially overcome these problems, very promising new systems have been proposed which allow patients and their caregivers to selectively stimulate the pudendal nerves to control voiding of the bladder. Such systems can eliminate the need for Foley catheters and are described, for example, in US Patent Publication No. 2014/0249595, the full disclosure of which is incorporated herein by reference.

While a significant advance, such pudendal nerve stimulation systems require surgical implantation of stimulation electrodes near the pudendal nerves, and the systems are not always successful and may require a second surgery for removal.

For these reasons, it would be desirable to provide improved systems and methods for controlling urination via stimulation of the pudendal nerves. It would be particularly desirable to provide apparatus and methods which would permit the testing of patients to determine the likelihood that such pudendal nerve stimulation will be effective and that the implantation sugary is warranted.

2. Description of the Background Art

US 2014/0249595, has been described above. See also US 2014/0058284; US 2014/0058588; and US 2014/0309550.

### SUMMARY OF THE INVENTION

Embodiments of the invention provide apparatus and methods for testing patients prior to the implantation of a nerve stimulation apparatus intended to control urination in patients who are unable or otherwise have a reduced ability to voluntarily control urination. Such patients can include paralyzed patients or any patient having a urinary dysfunction which impedes their ability to voluntarily control urination. Particular embodiments of the invention provide devices, systems, and methods to test patients to determine whether they would benefit from such implanted nerve stimulation therapies which allow the patient to control voiding of the bladder. Nerve stimulation therapies intended to control urination typically require implantation of electrodes to stimulate the pudendal nerve, a controller, and cable leads or wiring between the controller and the electrodes. By selectively stimulating the pudendal nerve with high frequency current and low frequency current, the bladder can be contracted and the urinary sphincter opened to allow control of urination without the need for having a Foley catheter residing the patient's urethra. Before implanting such devices, however, it would be desirable to determine if the patient is likely to benefit from the pudendal nerve stimulation. Various embodiments of the present invention allows the stimulation protocols to be mimicked using a temporarily introduced urethral catheter which can be used to both fill the bladder and to test the ability to control voiding of the bladder by applying stimulation current to the pudendal nerve to evoke or produce one or more physiologic actions involved in the urination process such as relaxation of urinary sphincter and contraction of the bladder. Embodiments of the invention are particularly useful for i) determining a selected system and/or protocol for controlling and/or optimizing urination via electrical stimulation of the pudendal nerves will be effective in control urination for a given patient; and ii) tuning or otherwise adjusting one or more characteristics of the electrical stimulation to optimize or otherwise enhance urinary function and/or control produced by the pudendal nerve therapy and for a particular patient. Such characteristics may include one or more of the frequency, current or voltage of the electrical stimulation used as well as the waveform of such stimulation.

In a first aspect, the present invention provides methods for screening a patient to determine the likelihood that the patient would benefit from pudendal nerve stimulation therapy. A catheter is temporarily introduced through the patient's urethra with a distal end of catheter entering the patient's bladder. The catheter is used to fill the bladder with a fluid, such as water or saline, and at least one electrode on the catheter is positioned within the urethra at a location selected to deliver current to the patient's pudendal nerve, typically within or near the patient's urinary sphincter. A low frequency current is delivered to the pudendal nerve using the electrode in order to contract the patient's bladder, and a high frequency current is delivered to the pudendal nerve to open the patient's urinary sphincter. The patient is considered likely to benefit from the pudendal nerve therapy if the delivery of the low frequency current and the high frequency current is able to successfully contract the bladder, open the urinary sphincter, and allow the fluid which has been introduced into the bladder to be voided in response to the delivered currents. In certain aspects of the methods, the catheter will further include a first pressure sensor allowing pressure within the bladder to be measured. Usually, the pressure sensor will be at or near the distal end of the catheter and will lie inside of the bladder. A first pressure sensor positioned at or near the distal end of the catheter allows a user to monitor bladder pressure as the bladder is being filled and to fill to a target pressure prior to delivering current to test voiding of the bladder.

Embodiments of the invention contemplate a variety of current delivery regimens may be used for assessing the effectiveness of the pudendal nerve stimulation in controlling urinary function. Typically, the high and low frequency currents will be delivered concurrently, though non currently delivery is also contemplated. The delivery of currents may be initiated simultaneously; however in one or embodiments, one may be initiated before the other and/or one can be stopped before the other. For example, according to one embodiment of a current delivery regimen, the high frequency current can be delivered first to relax the urinary sphincter. Then, the low frequency current is initiated and maintained for a select period of time to contract and void the bladder. The low frequency current is then stopped after the selected period of time but the high frequency is continued to keep open the urinary sphincter to allow urine to continue to flow due to any residual contraction of the bladder. Finally, the high frequency current is stopped to close the urinary sphincter.

One, two, or all three of bladder pressure, sphincter pressure and observed urine flow (qualitative and quantitative) may be used to assess the likely effectiveness of the therapy. In particular embodiments, one, two, or all three of these parameters may also be used to determine either or both an optimum high frequency and an optimum low frequency current which may be used in subsequent therapy to produce a selected and/or maximum urinary flow rate. For example, in one particular embodiment, one or both of the bladder pressure and urinary flow rate can be used to preselect a therapeutic low and/or high frequency current for a particular patient, i.e. the low or high frequency which will be used by the therapeutic device which is subsequently implanted. In another embodiment, one or both of the urinary flow rate and urinary sphincter pressure can be used for such preselection. In this way, the selection and/or settings of the implanted therapeutic device can be optimized for to provide maximum urinary function each patient before they have any implanted devices such as electrodes, wires controllers, etc.

In other embodiments of the invention, at least two electrodes will be positioned on the catheter to deliver current to the pudendal nerve. The first of the electrodes will be configured to deliver the low frequency current and a second of the electrodes will be configured to deliver the high frequency current.

In yet other embodiments of the present invention, the constrictive pressure exerted by the patient's urinary sphincter can be measured. Typically, the constrictive pressure is measured by a second pressure sensor on the catheter, and the second pressure sensor and the first and optionally second stimulating electrodes will usually be positioned on the catheter body so that each of the electrodes and the pressure sensor will lie close to or within the urinary sphincter surrounding the urethra when the catheter is present in the urethra with a distal end within the bladder. Further optionally, the catheter may include a balloon or other anchor structure near it's distil end, such as a balloon on a Foley catheter, so that the catheter may be positioned by retracting the catheter until the balloon or other anchor seats against or within the bladder neck. By selectively positioning the electrode(s) and the second pressure sensor, these elements will be properly positioned relative to the urinary sphincter when the catheter is in place.

In a second aspect, the present invention provides a catheter for screening a patient prior to pudendal nerve stimulation therapy, i.e. screening the patient for the effectiveness of pudendal nerve stimulation therapy prior to implantation of a therapeutic pudendal nerve stimulation device. The catheter includes a catheter body having a distal end, a proximal end, and a lumen for delivering a fluid from the proximal end to the distal end. The catheter body typically includes a first pressure sensor positioned at or near the distal end of the catheter body, typically positioned to measure pressure within the bladder when the catheter is in place within the urethra. The catheter will further include at least one electrode positioned on the catheter body at a location proximal of the distal end of the catheter. In particular the at least one electrode is positioned on the catheter so that it lies proximate the urinary sphincter when the catheter is properly placed within the urethra. In this way, the catheter can be used to deliver fluid to the bladder through the catheter lumen, measure pressure within the bladder using the first pressure sensor, and stimulate the bladder to cause contraction and/or open the urinary sphincter by properly applying low frequency and/or high frequency current through the electrode positioned to deliver current to the pudendal nerve, typically being near the urinary sphincter.

The screening catheter will typically apply both the high frequency and low frequency stimulation signals simultaneously or near simultaneously to mimic an actual therapeutic protocol where the sphincter is relaxed and the bladder contracted simultaneously to cause urination. In alternative embodiments, the high frequency current may be delivered first to open the sphincter followed, after a selected period, by the low frequency current to contract the bladder. In yet other alternative embodiments, the low frequency current may be delivered first to contract the bladder to generate greater pressure for more complete voiding followed after a selected period by the low frequency to open the urinary sphincter to allow voiding to commence.

In certain embodiments, the catheter of the present invention will further comprise at least a second electrode on the catheter body located near the first electrode, typically spaced proximally from the distal end of the catheter body. In these embodiments, the first and second electrodes will be usually be configured to be connected to different current sources capable of delivering low frequency current to contract the bladder and high frequency current to open the urinary sphincter. Suitable low frequency current to effect bladder contraction will be in the range from about 15 Hz to 50 Hz, with an amperage below 15 mA, and a voltage from 40 V to 60 V. Also in one or more embodiments the first and second electrodes may comprise pairs of bipolar electrodes which may be spaced apart a selected distance so as deliver current a selected depth into tissue to stimulate the pudendal nerve while minimizing effects on surrounding tissue.

In still other embodiments, the catheters of the present invention may further comprise a second pressure sensor on the catheter body at a location proximal of the distal end of the catheter body. Usually, the second pressure sensor will be located close to the first and optionally second electrodes, for example lying between the first and second electrodes, so that the first and second electrodes and the second pressure sensor may be positioned to lie adjacent or within the urinary sphincter when the distal end of the catheter is within the patient's bladder.

In still further embodiments, the catheters of the present invention may comprise a deployable anchor, such as a balloon similar to those found on conventional Foley catheters, positioned near the distal end of the catheter. The anchor is positioned on the catheter body so that the at least one electrode, and optionally the second electrode and pressure sensor, will lie adjacent to the pudendal nerve and urinary sphincter when the anchor is deployed at a neck of the bladder.

In a third aspect, the present invention provides systems comprising a catheter as described above in combination with a controller operably connectible to the electrode(s) to deliver a low frequency current to contract the patient's bladder and a high frequency current to open the patient's urinary sphincter. Usually, the controller will deliver the low frequency current to a first electrode and the high frequency current to a second electrode. The controller may further comprise or be connectible to one or more displays or screens configured to present data from the first pressure sensor to indicate bladder pressure and/or data from the second pressure sensor to indicate sphincter compression pressure or force.

Further details of these and other embodiments and aspects of the invention are described more fully below, with reference to the attached drawing figures.

### BRIEF DESCRIPTION OF THE DRAWINGS

FIG. 1 illustrates a prior art system for selectively stimulating the pudendal nerves to control urination taken from US Patent Publication No. 2014/0249595, the full disclosure of which has previously been incorporated herein by reference. In particular, FIG. 1 depicts a three-channel system for stimulating pudendal nerves or branches thereof. Pulse generator 10 is depicted as having three output channels. Wire leads 12 and 13 are attached to electrodes 14 and 15, which are placed about pudendal nerve 20. Electrode 15 is shown distal to electrode 14. In the context of a method comprising blocking the EUS and/or anal sphincter and stimulating or inhibiting contractions in the bladder and/or rectum, electrode 14 would be used to stimulate or inhibit bladder and/or rectal contractions, while electrode 15 would be used to block the EUS or anal sphincter. As needed, wire lead 16 is attached to another electrode (not shown in FIG. 1) that is placed about the pudendal nerve on the other side of the body to block the pudendal nerve on the opposite side. Pulse generator 10 is shown as implanted beneath skin 40. Output parameters of the pulse generator 10 can be controlled via a wired interface, but preferably is controlled by wireless transmission, which can be carried any suitable wireless protocol, such as radio frequency, IEEE 802.11a/b/g, Bluetooth, etc. Thus, an external controller 30 is depicted for communicating with the pulse generator 10. External controller 30 is depicted as having a display 32, such as an LCD, LED or OLED display, and a keypad 34 for entering data into the external controller 30. External controller is depicted as sending a wireless transmission 36 to pulse generator 10, though in another embodiment, data can be transferred both to the pulse generator 10 from the external communicator 30 and vice-versa, to permit monitoring of one or more parameters of pulse generator 10, including, without limitation, output signal characteristics (e.g., frequency, amplitude, etc. as outlined above) and battery strength. Activity of pulse generator 10 and external controller 30 typically is microprocessor controlled and software/firmware installed onto the pulse generator 10 and external controller 30 hardware may be used to implement the described tasks, and to provide, for example and without limitation, a GUI (graphical user interface) for the display 32, which facilitates use of the system. Both pulse generator 10 and external controller 30 may comprise any suitable electrical and electronic components to implement the activities, including, microprocessors, memory (e.g., RAM, ROM. Flash memory, etc.), connectors, batteries, power transformers, amplifiers, etc.

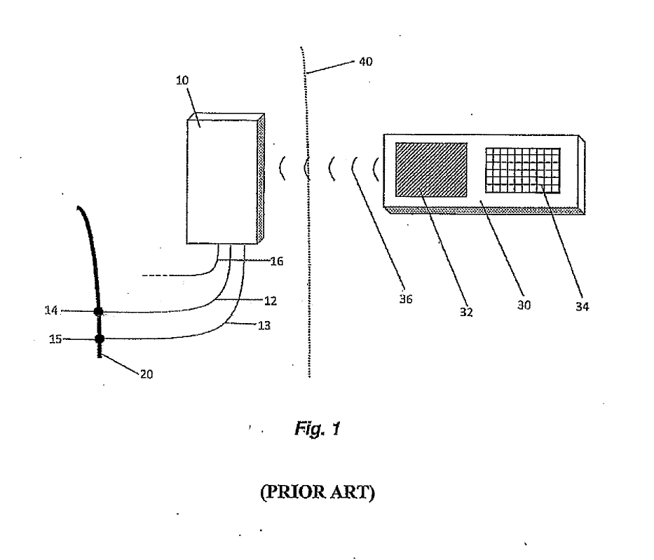

FIG. 2 illustrates an exemplary embodiment of a test catheter constructed in accordance with the principles of the present invention.

FIG. 3 illustrates the test catheter of FIG. 2 placed in a patient's urethra to perform a test protocol in accordance with the methods of the present invention.

FIG. 3a is an enlarged view of FIG. 3 illustrates the positioning of the test catheter electrodes within the patient urinary sphincter to stimulate the patient's pudendal nerve.

FIGS. 4a and 4b illustrate an embodiment of the test catheter having a second lumen for the flow of fluid from the bladder to outside the patient.

FIG. 5 illustrates an embodiment of bipolar electrodes for use as pudendal nerve stimulation electrodes for use with an embodiment of the test catheter.

### DETAILED DESCRIPTION OF THE INVENTION

Embodiments of the invention provide devices, systems and methods for testing patients incapable of and/or having reduced voluntary control of urinary function to see if the tested patient would benefit from pudendal nerve stimulation therapies which allow the patient to control voiding of the bladder. Such patients may include those who are paralyzed due to spinal cord injury and/or patients who have multiple sclerosis or other motor neuron disease. Particular embodiments of the invention provide apparatus and methods of the present invention allow testing of bladder stimulation protocols which mimic those provided by certain implantable devices to provide bladder control, such as those described in US Patent Publication No. 2014/0249595, previously incorporated herein by reference, and co-pending, commonly owned U.S. Provisional No. 62/280,639, filed Jan. 19, 2016, the full disclosure of which is incorporated herein by reference.

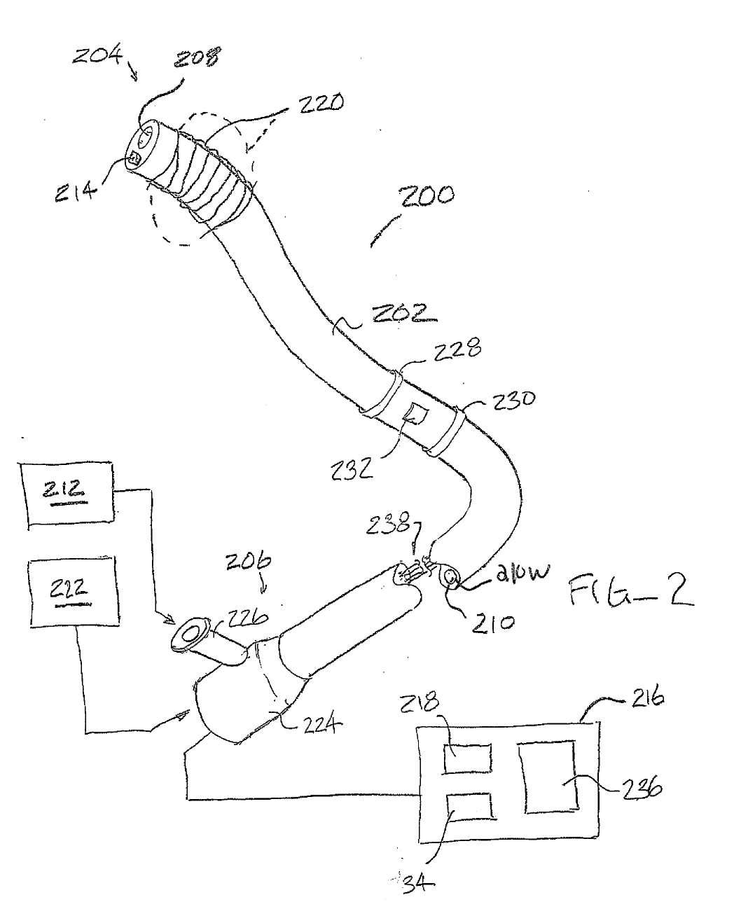

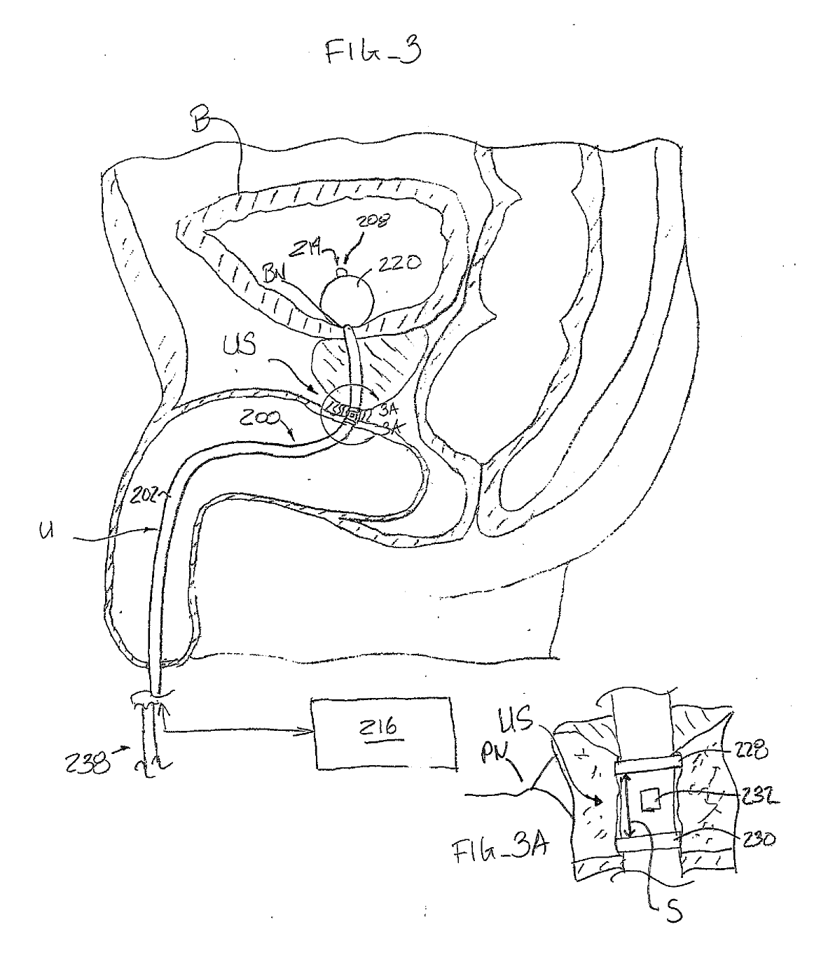

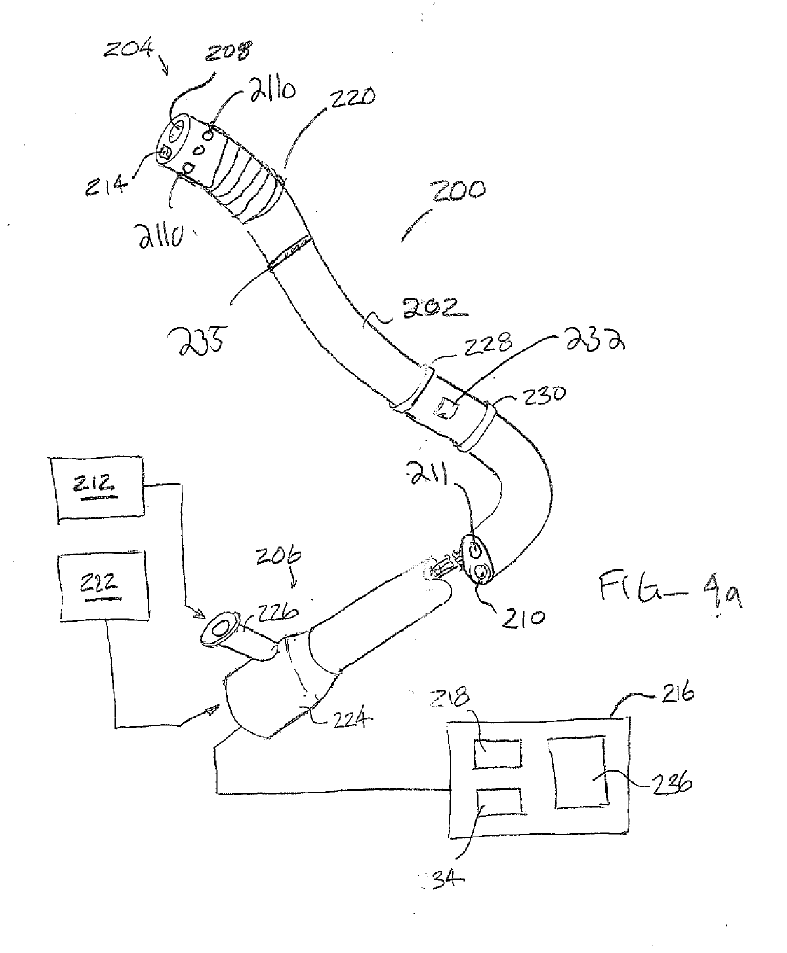

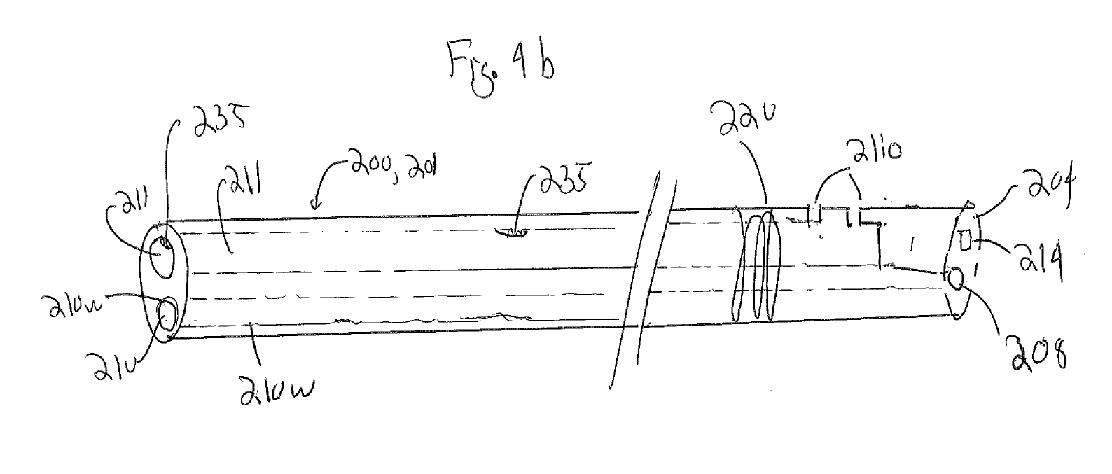

Referring now to FIGS. 2, 3a and 4a-4b and 5 an embodiment of a test catheter 200 comprises a catheter body 202 having a distal end 204 and a proximal end 206 and at least two electrodes 228 and 230 for stimulating the pudendal nerve PN to evoke otherwise produce physiologic responses involved in the urination process such as contraction of the bladder and relaxation of the urinary sphinter. The catheter body may be fabricated from various flexible biocompatible polymer known in the art including, for example, HDPE (high density polyethylene), LDPE (low density polyethylene), Pebax® polymer, PTFE, silicone, polyurethane, or other elastomer known in the catheter art. A lumen 210 having a port 208 at the distal end of the catheter body 202 is fluidically connectable to a fluid source 212 through a port 226 on a proximal hub 224 of the catheter. As shown in FIGS. 4a and 4b, in alternative or additional embodiments, the test catheter 200 may also include a second lumen 211 for the flow of fluid from the patient's bladder B, to outside the body to facilitate voiding and allow for measurement of urinary flow rates as is described in more detail below. In various embodiments the fluid source 212 may correspond to a pressure-controlled pump, a hanging fluid bag (for example a bag of premixed saline solution), or any other conventional fluid source which can deliver saline, water, or other suitable pressurizing liquid to the patient's bladder through the lumen 210 when the distal end 204 the catheter 200 is present within a bladder B, as is illustrated in FIG. 3a described hereinafter.

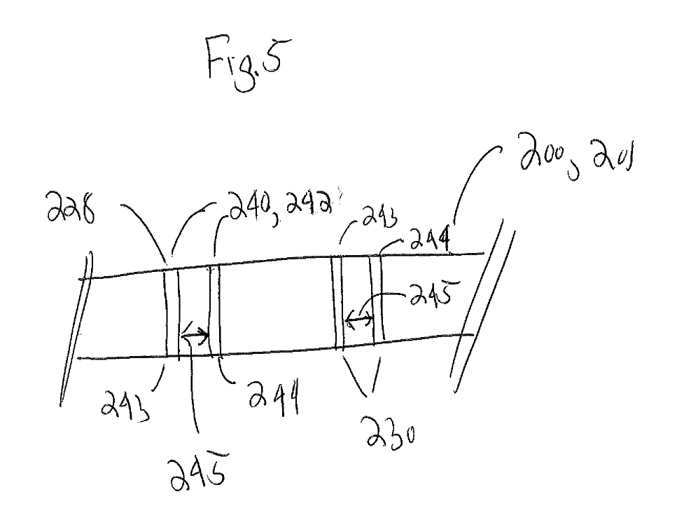

Referring now to FIGS. 3a and 5, electrodes 228 and 230 many comprise various biocompatible conductive metals and other conductive materials known in the medical device and electrophysiology arts. In various embodiments electrodes 228 and 230 may correspond to various ring electrodes and may have a selectable width e.g. 1 to 10 mms selected so to achieve a desired depth of penetration through urinary sphincter tissue to the pudendal nerve. The spacing S between electrodes 228 and 230 is desirably selected to position the electrodes on the interior sides of the urinary sphincter US as is shown in FIG. 3a.

As shown in FIG. 5, in alternative or additional embodiments, pudendal nerve stimulation electrode 228 and 230 may correspond to individual pairs 242 of bipolar electrodes 240. With each pair 242 of bipolar electrodes including a first and second electrode 243 and 244 though which alternating or other current may flow between. The distance or gap 245 between each individual electrode 243 and 244 of the bipolar pair 240 may be selected to control the depth of penetration of the stimulation current flow between them to selectively target the pudendal nerve while avoiding any surrounding tissue. Longer distances tend to produce deeper penetration while shorter distance less. In various embodiments, gap 245 can range between about 1 to 5 mm, with greater and shorter gaps contemplated. The gap may also be selected depending on whether the current is high frequency or low frequency current as is described herein.

According to one or more embodiments, the catheter body 202 and/or lumen 210 or other lumen 211 may be sufficiently rigid (e.g., radial rigidity or stiffness) and/or may have sufficient hoop strength to prevent fluid pressure in an individual lumen from appreciably affecting pressure measurement of bladder pressure and/or constrictive pressure of the urethra sphincter by catheter 200 using sensors sensor 214 and 232 or another sensor. Appreciably meaning an effect on pressure below about 10%, more preferably below about 5% and still more preferably below about 1%. More specifically, the one or more lumens of catheter 200 (which here means the lumen walls as well) are desirably sufficiently rigid so to minimize or prevent the transmission of force from a fluidic pressure inside a lumen to outside the catheter which may affect a measurement by catheter 200. Desirably, the transmitted force is less than 0.2 lbs, more desirably, below 0.05 lbs and still more desirably below 0.01 lbs of force. Similarly, lumens 210, 211 also sufficiently rigid so as to minimize the transmission of a fluidic pressure from lumen to the other which may affect a pressure measurement and/or constrict an adjacent lumen to appreciably affect a flow rate through the adjacent lumen. In other words, the lumens are sufficiently rigid to prevent hydrostatic pressure cross-talk from one lumen to the next and prevent a pressure in one lumen from constricting an adjacent lumen. They are also sufficiently rigid to maintain their shape from the exertion of a force an adjacent lumen or when the catheter body is bent when positioned in the patient's urinary tract. Preferably, though, the catheter 200 and catheter body 201 as a whole will remain sufficiently compliant to be advanced and manipulated in and through the intended patient anatomy for the clinical use scenarios. In specific embodiments, the catheter lumens 210 and 211 have sufficient stiffness or hoop stress such that any change in pressure or flow rate in one lumen resulting from a pressure or change in pressure in an adjacent lumen will remain at or below, preferably below, about 5%, preferably 2% and still more preferably below 1%. In various embodiments, such stiffness or hoop strength can be achieved by any one or more of the following: (1) choice of catheter materials, (2) catheter/lumen dimensions, (3) use of a reinforcing braid (internal external to the lumen), and/or (4) an internal re-enforcing lumen. In various embodiments, the radial rigidity (also described herein as radial stiffness), of any one of lumens 210, 211 or other lumen of catheter 200 can be in the range of about 1 to about 100 N/mm, more preferably in a range of about 20 to about 100 N/mm and still more preferably in a range of about 50 to about 100 N/mm with specific embodiments of 5, 10, 20, 25, 30, 40, 45, 50, 55, 60, 70, 75, 80, 90 and 95 N/mm; whereas the hoop strength can be in a range of about 0.25 to 5 lbs, more preferably about 0.5 to 5 lbs, and still more preferably about 1 to 10 lbs, with specific embodiments of 0, 5, 1, 2, 2, 5, 3, 4, 5, 6, 7, 8 and 9 lbs of force.

In one or more embodiments, catheter 200 will typically also comprise a first pressure sensor 214 located at or near the distal end 204 of the catheter body 202, though it should be appreciated that other positions on the catheter body for the pressure sensor are also considered. The first pressure sensor 214 will typically be a solid state pressure transducer (e.g., various solid state strain gauges known in the art including Mems based strain gauges) suitable for measuring pressures within the bladder, typically in a range from 0 to 50 mmHg though other ranges are also considered. Normal bladder pressure is in the range from 0 to 10 mmHg or sometimes 0 to 20 mmHg, but the methods of the present invention may rely on delivering and measuring pressure above the normal bladder pressure. Bladder pressure read by the transducer 214 may be displayed on a readout 218 on controller 216 or may wireless transmitted to an external display using Bluetooth® or BluetoothLE® or other wireless communication protocol and/or wireless means. The readout 218 may be a dedicated LCD, LED, or other numeric or analog readout, or could be part of a display screen, optionally a touch screen display. The catheter 200 will optionally include a distal anchor, such as inflatable balloon 220 which will be positionable in a patient's bladder in order to stabilize the catheter body 202 and properly position electrodes 228 and 230 at or near the patient's urinary sphincter, as will be described in greater detail with respect to FIG. 3a below. A second pressure transducer 232 will often be provided near the electrodes 228 and 230 in order to allow measurement of the contraction pressure of the urinary sphincter. The pressure sensors 214 and 232 and the electrodes 228 and 230 will be connected by wires or cables 238 passing through the catheter body and connecting the electronic components to the controller 216. They may also be wireless devices configured to communicate with controller 216 by BlueTooth® or other wireless protocol. The balloon 220 will be inflated by a conventional inflation source 222 of the type normally used with Foley catheters. Sphincter pressure measured by the second pressure transducer 232 may be shown on a second display element 234 on the controller 216 or may also be shown on a conventional touch screen or other display on the controller. The controller 216 will usually also include a user interface 236. According to various embodiments, user interface 236 may be a touch screen (which can be the same touch screen to display all data) a keyboard, joy stick, or any other conventional user interface which may be directly or wirelessly coupled to the controller.

Referring now to FIG. 3, the catheter 200 of FIG. 2 may be introduced through the patient's urethra U so that the anchor balloon 220 may be positioned within the patient's bladder B so that it rests above or within the bladder neck BN. The dimensions for the catheter may be selected such that electrodes 228 and 230 and second pressure sensor 232 will be positioned generally within the patient's urinary sphincter US so as to stimulate the Pudendal Nerve PS, as best shown in FIG. 3a so as to sit.

In one or more embodiments, accommodations in the length and other dimensions of catheter 200 and its components can be made for the sex of the patient. For example, in the case of a catheter 200 adapted for the male urinary tract, second sensor 232 for measuring urethra constrictive pressure may be positioned on the catheter body 201 approximately 2.5 cm to 3.5 cm proximal to the distal end 204 of the catheter and extends approximately 2.0 cm of urethral length. In the female version of the catheter sensor 232 is located approximately 1.8 to 3cm proximal to distal catheter end 204, and extends approximately 1.5 cm of urethral length.

In the position shown in FIG. 3, the catheter 200 may be used to first introduce a fluid, such as water or saline, into the bladder B in order to fill and pressurize the bladder to a desired target level, typically from about 10 mmHg to about 45 mmHg with a preferred range from about 10 to about 20 mmHg. In related embodiments, catheter 200 may be used to fill the bladder to a target volume, for example in a range from about 50 to 200 ml. As used herein the term “about” means within ±10% of a stated value for a parameter, measurement, dimension, characteristic, physical property and the like. Once the bladder B is filled to desired pressure and/or with a desired volume of fluid, a low frequency bladder contraction current, typically having a frequency from about 15 Hz to about 50 Hz, may be applied through the first electrode 228 so as to stimulate the Pudendal Nerve PN cause contraction of the bladder. Simultaneously or near simultaneously (e.g., in this case within about 0.2 seconds), a high frequency urinary sphincter relaxation current is applied by the second electrode 230 to cause relaxation and opening of the urinary sphincter. In some embodiments, the high frequency current may be initiated at a selected time period before the low frequency current (e.g., about 0.2 to 2 seconds) so as to allow the urinary sphincter to relax and completely open up before the bladder contracts from the low frequency current so that fluidic resistance in the urethra to bladder contraction is minimized before the bladder begins to contract. Typically, the high frequency current has a frequency in the above 4 kHz with an amperage below about 15 mA and a voltage in the range from 40 V to 60 V, will be Specific ranges for the high frequency current can include 4 to 10 kHz, 4 to 15 kHz and 4 to 20 kHz with still other ranges contemplated. When the high and low frequency currents are applied, contraction of the bladder and opening of the urinary sphincter allows the patient to urinate (also referred to herein as voiding) through an annular urethral space surrounding the catheter 200.

In alternative or additional embodiments, a second high frequency current, typically above 1 kHz may be delivered to the pudendal nerve by catheter 200 so as to block or otherwise attenuate any tingling, burning, numbness or related sensation (known as paresthesia) that is caused by the current delivery to the pudendal nerve by electrodes 228 and 230. This current, described herein as a paresthesia inhibiting current may delivered by either or both of electrodes 228 or 230 or may be delivered by a third electrode on catheter 200 (not shown) or external to it. Desirably, the parathesia inhibiting current is generated by a different current source that that used to generate the first two currents (e.g., the bladder contraction and sphincter relaxation currents). In various embodiments, the parathesia inhibiting current may have a frequency in a range from 1.5 kHz to 100 kHz, more preferably in a range from about 3 kHz to 20 kHz. In one or more embodiments the wave form of the parathesia inhibiting current may correspond to bi-phasic pulses having a pulse width in a range from 10 μseconds to 333 microseconds and more preferably in a range from about 30 to 35 μseconds. The amplitude can be in can be varied from about 1 mA to about 4 mA with a nominal value of about 2.5 mA. These and other characteristics of the parathesia inhibiting current can be tuned or fine tuned or otherwise adjusted based on one or more of feedback from the patient on perceived parathesia and/or measurement of bladder pressure, urinary sphincter constrictive pressure and urinary flow rates in a similar fashion to adjustment of the current to contract the bladder and relax the urinary sphincter. In this way, embodiments of the invention can be used to optimize the parathesia inhibiting current before a pudendal nerve stimulation device is implanted so that the patient has a better outcome than if pudendal nerve stimulation device was preprogramed with a standard parathesia inhibiting current.

Referring now to FIGS. 4a and 4b. In alternative or additional embodiments, the catheter 200 can be provided with an additional lumen or other channel 211 to facilitate voiding or urination through the lumen 211 while the catheter 200 is in positioned in the desired location in the patient's urinary tract. Lumen 211 can include one or more distal opening 211o for the flow of fluid into the catheter from the bladder. Desirably, openings 211o are positioned a selected distance proximal to the distal end 214 of the catheter so that openings 211o, lie slightly above the bladder neck and anchor device 220 to facilitate flow of fluid into the catheter and complete voiding of the bladder. Again, as is discussed herein lumen 211 can have sufficient radial stiffness (e.g., through the use of a reinforcing braid) so that it maintains its shape when catheter is bent and/or subjected to fluidic pressure from lumen 210. In use, embodiments of catheter having dedicated lumen 211 for fluid flow out of the bladder, facilitate more accurate measurement of urinary flow rates while the pudendal nerve is being stimulated by electrodes 230 to evoke urination as the all of the urinary flow goes through the catheter and flow rates or total volumes may be measured. The measurement can be done manually (by timing and collecting the voided fluid) or using an flow sensor which may be an external sensor positioned over the catheter or at the proximal end of the catheter. According to some embodiments, catheter 200 may include its own flow sensor 235 which may be positioned in or around lumen 211 or catheter body 20. Flow sensor 235 may also may be operatively coupled to controller 216 to information on urinary flow to the controller. Flow sensor 235 may correspond to various electromagnetic, optical, aneometric and acoustic flows sensors known in the art including various solid state flow sensors. Further in particular embodiments, the measured urinary flow rates may be used to adjust one more characteristics of the high and low frequency current including for example, amplitude (either of the voltage and/or current), frequency, pulse width, as is described herein. Suitable target urinary flow rates for men and women are shown in Table 1. In various embodiments, the fluidic resistance in lumen 211 (including that for an individual catheter 210) at flow rates and bladder pressures corresponding to those typical during urination can be premeasured and stored in a memory device shipped with the catheter and configured to be operative coupled to controller 216. Alternatively, they may be calculated for a standard diameter lumen 211 and stored in the controller 216. Such fluidic resistance can then be used to convert the fluid flow rate through lumen 211 that results from evoked bladder voiding to rates which would be expected through the urethra were catheter 200 not positioned in the patient urinary tract

As discussed herein In these and related embodiments, first lumen 210 (which includes the lumen wall 210w) can have sufficient radial rigidity or otherwise reinforced so that when the lumen 210 is subjected to various pressures during fluid flow to fill the bladder, it does not cause any appreciable radial deformation of the diameter of the additional lumen so as to reduce the urinary flow rate out through the second (urinary outflow) lumen by an appreciable amount (e.g., less than 10% more preferably less than 5% and still more preferably less than 1%). Additionally, the second pressure transducer 232 is desirably configured to measure a lessening of the urinary sphincter pressure as the sphincter relaxes. Observation/measurement of the passage of urine and optionally, the lessening of the sphincter pressure will indicate that the patient responds favorably to the pudendal nerve stimulation and is a good candidate for a full implantation procedure. This and other information on the physiologic responses to the delivered high and low frequency or other currents can be used as criteria to assess the effectiveness of the pudendal therapy.

In one or more embodiments, various information collected using catheter 200 or other measurement means relating to the produced physiologic response from the pudendal nerve therapy can be used as criteria for assessing the effectiveness of the therapy in treating the patients urinary dysfunction (e.g., being able to initiate and control urination). Such information may also be used to adjust one or more characteristics of the high and low frequency currents so as to optimize a pudendal nerve therapy for a particular patient. According to various embodiments, such criteria can include one or more of urinary flow rates, decreases in urinary sphincter pressure and increases in bladder pressure. For the case of urinary flow, minimal flow rates within 0 to 30% of the values shown in Table 1 may be used. While in the case of urinary sphincter pressure, a reduction in pressure by about 50, 60, 70, 75, 80, 90 or 95% (with other values also contemplated) may be used. Further for the case of bladder pressure, an increase in the amount of about 10, 20, 30, 40, 50, 60. 70, 80, 90, 95% (with other values also contemplated) may be used.

TABLE 1
Average Urinary Flow Rates for Men and Woman by Age.

| Age Range | Male | Female |
|---|---|---|
| 8 to 13 | 12 ml/sec | 15 ml/sec |
| 14 to 45 | 21 ml/sec | 18 ml/sec |
| 46 to 65 | 12 ml/sec | 18 ml/sec |
| 66 to 80 | 9 ml/sec | 18 ml/sec |

In particular embodiments, one or all three of urinary/void flow rate, decrease in urinary sphincter pressure and increase in bladder pressure may be used to preselect both the high frequency and low frequency currents to be used in a subsequently implanted pudendal therapy device. In particular, the high and low frequencies may be “tuned” (grossly adjusted) or “finely tuned” (finely adjusted) while observing or tracking changes in urinary flow rate, so as to identify those settings which result in a selected and/or maximum urinary flow rate and or shortest duration of urination. Specific adjustments may be made in one or more characteristics of the high and low frequency currents including of the frequency, current or voltage of the wave as well as the shape of the wave. In particular adjustments may be made in in the peak amplitude of the current or voltage as well as the RMS amplitudes. Also different waveforms may employed including for example sine wave, square wave and saw tooth waves. Also, in one or more embodiments, the waveform may in the form of biphasic pulses with a selectable pulse width, for example 1 to 100 ms.

Gross adjustments may incorporate changes in the range of about 5 to about 25%, while fine adjustments may those less than about 5%. For example, in one particular embodiment, one or of the bladder pressure and urinary flow rate can be used to tune and fine tune the low frequency current to a particular patient to produce a selected and/or optimized urinary flow rate and/or optimized micturition sequence (e.g. timing of bladder contraction and urinary sphincter relaxation) for a particular patient. In another embodiment, one or both of the urinary flow rate and urinary sphincter pressure can be used to tune or fine tune the high frequency current to produce selected and/or optimized urinary flow rate and/or optimized micturition sequence (e.g. timing of bladder contraction and urinary sphincter relaxation) for a particular patient. In this way, candidates for pudendal nerve stimulation therapy to enable or improve urinary function can not only be selected but further, the urinary function achieved by embodiments of pudendal nerve stimulation therapy can be optimized for each patient before they have any implanted devices such as electrodes, wires controllers, etc. This in turn improves the ultimate clinical outcomes for the candidate patients for pudendal nerve stimulation therapy and also reduces the risk of morbidity and mortality from the necessity of having to remove and/or re-implant electrodes and other components of pudendal nerve stimulation system.

The foregoing description of various embodiments of the invention has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric applications as well as various veterinary applications. Also those skilled in the art will recognize, or be able to ascertain using no more than routine experimentation, numerous equivalents to the specific devices and methods described herein. Such equivalents are considered to be within the scope of the present invention and are covered by the appended claims below.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Hence, the scope of the present invention is not limited to the specifics of the described embodiments, but is instead limited solely by the appended claims.

What is claimed is:

1. A method for screening a patient for pudendal nerve therapy to treat a urinary dysfunction in the patient, the method comprising:
introducing a catheter through the patient's urethra into the patient's bladder, the catheter including at least one electrode and at least one lumen;
positioning the at least one electrode on the catheter at a position in the urethra to deliver current to the patient's pudendal nerve;
delivering fluid into the patient's bladder through the catheter;
delivering current through the at least one electrode to the pudendal nerve, the current comprising at least one of a low frequency current to contract the patient's bladder and a high frequency current to open the patient's urinary sphincter;
measuring information on a physiological response produced in response to the delivered current; and
utilizing the information on the produced physiological response to assess the patient's response to the delivered currents.

2. The method of claim 1, wherein the at least one electrode comprises at a first and second electrode which are positioned to deliver current to the pudendal nerve.

3. The method of claim 2 wherein the first electrode delivers the low frequency current and the second electrode delivers the high frequency current.

4. The method of claim 3, wherein the catheter is positioned so that each of the first and second electrodes are positioned near the urinary sphincter.

5. The method of claim 1, wherein the catheter includes at least two lumens, a first lumen for filing the bladder and a second lumen for voiding of fluid form the bladder when the low frequency is delivered to contract the bladder.

6. The method of claim 1, wherein the bladder is filled to a target pressure.

7. The method of claim 1, wherein the target pressure is in a range from about 10 to 45 mmHg or about 10 to 20 mmHg.

8. The method of claim 1, wherein the bladder is filled to a target volume.

9. The method of claim 8, wherein the target volume is in a range from about 50 to 200 ml.

10. The method of claim 1, wherein the high and low frequency currents are delivered near simultaneously.

11. The method of claim 1, wherein delivery of the high frequency current is initiated before that of the low frequency current.

12. The method of claim 1, wherein the low frequency current is in a range of about 15 Hz to 50 Hz.

13. The method of claim 1, wherein the high frequency current is in a range of about 4 to 10 kHz.

14. The method of claim 1, wherein the catheter is positioned to block the opening of the patient's bladder such that when the bladder is contracted, voided fluid flows substantially through a lumen in the catheter.

15. The method of claim 14, further comprising measuring a flow rate of voided fluid flowing the catheter.

16. The method of claim 14, wherein the catheter includes an inflatable balloon or other means for blocking the opening of the patient's bladder.

17. The method of claim 1, wherein the fluid flowing through the catheter in or out of the bladder does not substantially affect measurement of the information on the produced physiologic response.

18. The method of claim 17, wherein the information not affected includes at least one of bladder pressure, urinary sphincter pressure or urinary flow rate.

19. The method of claim 17, wherein the lumen of the catheter has sufficient radial rigidity such that a force from a pressure of fluid within the catheter is not substantially transferred outside of the catheter.

20. The method of claim 1, wherein the information on the produced physiological response is used to adjust a characteristic of the high frequency or low frequency current.

21. The method of claim 20, wherein the characteristic is a frequency, magnitude or waveform shape of the high frequency or low frequency current.

22. The method of claim 20, wherein the adjustment comprises a tuning or fine tuning of at least one of the high frequency or low frequency current.

23. The method of claim 22, wherein the fine tuning comprises adjustment of the characteristic by an amount less than about 5 percent.

24. The method of claim 22, wherein the tuning comprises adjustment of the characteristic in amount in a range of about 5 to about 25 percent.

25. The method of claim 22, wherein the characteristic of the high frequency or low frequency current is used to enable or improve the patient's urinary function.

26. The method of claim 1, wherein the urinary dysfunction is a reduced ability to voluntarily control urination.

27. The method of claim 1, wherein the measured information is a bladder pressure.

28. The method of claim 27, wherein the bladder pressure is measured using a pressure sensor.

29. The method of claim 27, wherein the measured information further comprises a constrictive pressure of the urinary sphincter.

30. The method of claim 29, wherein the bladder pressure and the urinary sphincter constrictive pressure are simultaneously measured.

31. The method of claim 28, where the bladder pressure sensor is positioned on the catheter or a distal end of the catheter.

32. The method of claim 28, wherein the catheter is advanced to position the pressure sensor inside the bladder.

33. The method of claim 1, wherein the measured information is a constrictive pressure of the urinary sphincter.

34. The method of claim 33, wherein the constrictive pressure is measured using a pressure sensor.

35. The method of claim 34, wherein the pressure sensor is positioned adjacent the urinary sphincter.

36. The method of claim 1, wherein the measured information comprises a urinary flow rate.

37. The method of claim 36, wherein a characteristic of the high frequency or low frequency current is adjusted to achieve a target urinary flow rate.

38. The method of claim 37, wherein the characteristic is a frequency or magnitude of the high frequency or low frequency current.

39. The method of claim 37, wherein the target urinary flow rate is in a range from about 9 to 21 ml per minute for a male patient and 15 to 18 ml per minute for a female patient.

40. A catheter for screening a patient for pudendal nerve therapy, the catheter comprising:
a catheter body having a distal end, a proximal end, and a first lumen for delivering a fluid from the proximal end to the distal end, the lumen having a distal opening;
a first pressure sensor positioned on the catheter body near the distal end of the catheter body to measure the patients bladder pressure;
a second pressure sensor positioned at a location proximal to the distal end of the catheter body to measure the patients urinary sphincter pressure; and
at least one electrode positioned on the catheter body at a location proximal to the distal catheter end to deliver current to the pudendal nerve,
wherein the catheter body is configured to be advanced through the patient's urethra to position the first pressure sensor and the distal opening of the lumen within the patient's bladder; and
wherein when catheter is so positioned, the first lumen is configured to allow the flow of fluid into the patient's bladder with no appreciable effects on a concurrent measurement of the patient's bladder pressure or urinary sphincter pressure by the first or second pressure sensors.

41. The catheter of claim 40, where the first lumen has sufficient radial rigidity or hoop strength to minimize the transfer of force from fluid pressure inside the lumen to outside the catheter body.

42. The catheter of claim 41, wherein the first lumen includes a reinforcing braid configured to minimize the transfer of force from fluid pressure inside the lumen to outside the catheter body.

43. The catheter of claim 40, wherein the catheter body includes a second lumen for the flow of fluid from the bladder to outside the patient's body.

44. The catheter of claim 40, wherein a radial rigidity of the first lumen or second lumen is in a range from about 50 to 100 N/mm.

45. The catheter of claim 40, wherein the catheter is adapted to be positioned in a male urinary tract.

46. The catheter of claim 45, wherein the second sensor has a length of about 2 cms and is positioned about 2.5 to 3.5 cms from the distal end of the catheter.

47. The catheter of claim 40, wherein the catheter is adapted to be positioned in a female urinary tract.

48. The catheter of claim 47, wherein the second sensor has a length of about 1.5 cms and is positioned about 1.8 to 2.8 cms from the distal end of the catheter.

49. The catheter of claim 40, wherein the catheter body comprises an elastomer, silicone, or polyurethane, PTFE, polyethylene or PET.

50. The catheter of claim 40, wherein the at least one electrode comprise a pair of bipolar electrodes.

51. The catheter of claim 50, wherein the pair of bipolar electrodes are positioned a selected distance apart so as to target a depth of the delivered current in tissue to the pudendal nerve.

52. The catheter of claim 40, further comprising at least a second electrode on the catheter body positioned at a location proximal of the distal end, wherein the first and second electrodes are configured to be connected to different current sources.

53. The catheter of claim 52, wherein each of the first and second electrodes and the second pressure sensor are positioned so that they will lie within the patient's urinary sphincter when the distal end of the catheter is within the patient's bladder.

54. The catheter of claim 40, further comprising a deployable anchor positioned on the catheter body so that the at least one electrode will lie adjacent the pudendal nerve when the anchor is deployed at a neck of the bladder.

55. The catheter of claim 54, wherein the deployable anchor is positioned near the distal end of the catheter body.

56. The catheter of claim 54, wherein the deployable anchor comprises an inflatable balloon.

57. A system for screening a patient for pudendal nerve therapy to treat a urinary dysfunction in the patient, the system comprising:
the catheter of claim 52; and
a controller connectable to the first and second electrodes to deliver a low frequency current to contract the patient's bladder to the first electrode and a high frequency current to open the patient's urinary sphincter to the second electrode.

58. The system of claim 57, wherein the controller further comprises a pressure display connectable to at least one of the first pressure or second pressure sensors to display at least one of bladder pressure or urinary sphincter contraction pressure.

59. The system of claim 57, wherein the controller is further configured to deliver a paresthesia inhibiting current to inhibit or reduce paresthesia resulting from the low frequency or high frequency current.

60. The system of claim 59, wherein the catheter body includes a third electrode for delivery of the paresthesia inhibiting current.

61. The system of claim 59, wherein the paresthesia inhibiting current has a frequency in the range of about 3 kHz to 20 kHz.
